(19) United States
(12) Patent Application Publication
(10) Pub. No.: US 2015/0336872 A1
(43) Pub. Date: Nov. 26, 2015

(54) PROCESS FOR THE PURIFICATION OF CARBOXYLIC ACIDS

(71) Applicant: THYSSENKRUPP AG, Essen (DE)

(72) Inventors: Markus Fritsch, Leipzig (DE); Joachim Schulze, Soest (DE); Christoph Lütge, Unna (DE); Zeljko Knez, Maribor (SI)

(21) Appl. No.: 14/654,398

(22) PCT Filed: Dec. 20, 2013
(86) PCT No.: PCT/EP2013/003895

§ 371 Date: Jun. 19, 2015

(30) Foreign Application Priority Data
Dec. 21, 2012 (EP) 12008534.5

(51) Int. Cl. C07C 51/493; G01N 30/72; G01N 30/74; B01J 19/24; G01N 30/02

(52) U.S. Cl. CPC C07C 51/493; B01J 19/24; G01N 30/02; G01N 30/74; G01N 30/72; B01J 2219/24

(57) ABSTRACT

A process for the purification of carboxylic acid having a chain length from one to five carbon atoms is disclosed herein. The process includes transforming the carboxylic acid to its monoester and/or its diester, and processing the mono- and/or the diester of the carboxylic acid by subcritical or supercritical fluid chromatography using a subcritical or supercritical mobile phase.

### CROSS-REFERENCE TO RELATED APPLICATIONS

This application is the national phase of, and claims priority to, International Patent Application No. PCT/EP2013/003895, filed Dec. 20, 2013, which designated the U.S. and which claims priority to European Patent Application No. EP 12008534.5, filed Dec. 21, 2012. These applications are each incorporated by reference herein in their entireties.

### BACKGROUND

1. Field of the Invention

The invention relates to a process for the purification of carboxylic acid. Also a device for the execution of the process, and a use for the process according to the invention are taught. The isolation of carboxylic acids, which are not able to be separated using distillation or which can only be separated with difficulty, is extremely complex.

2. Description of the Related Art

Key to the industrial application of carboxylic acids, which are produced using various microorganisms in the fermentation of carbohydrate-containing substrates, is the economic viability and efficiency of separating and purifying the desired carboxylic acid from these aqueous fermentation solutions which, as well as containing carboxylic acid or carboxylic acid salts, also contain other organic acids, other by-products of fermentation, microorganisms and their components, as well as residues of the substrates, such as sugar. These impurities interfere with the subsequent processing of the carboxylic acids produced. As an example, lactic acid is polymerised to form polylactic acid which is used to produce biodegradable plastics. Extremely pure monomer must be used for this purpose to ensure that a high degree of polymerisation is achieved for the lactic acid. This has been known for some time and covered, for example, in J. Dahlmann et al, British Polymer Journal, Vol. 23 (1990), Page 235. 240.

Succinic acid, for example, is known to be similar. The qualities of the succinic acid produced can be differentiated by classifying them in terms of technical quality with a succinic acid content of at least 97 Ma-% and with a succinic acid which is specially suited for use in polymerisation (polymer grade) with a content of at least 99.5 Ma-%.

A number of patents describe the production of succinic acid from fermentation solutions, including:
extractive processes using extracting agents, such as tributylamines, trialkylamines, olefins, various alcohols, and aromatic hydrocarbons;
processes using calcium hydroxide and sulphuric acid, wherein gypsum is produced as the by-product;
processes using electrodialysis;
thermal methods, such as fractional distillation or thermally staged chromatography;
high-pressure extraction using CO₂; and
membrane processes, such as reverse osmosis and other filtration processes
   wherein the coupling of these processes and the supplementing of them with further steps in line with prior art are discussed. These types of processes are described, amongst others, in patent specifications DE 69821951 T2 ; DE 69015233 T2 ; DE 69015019 T2 ; DE 69006555 T2 ; DE 69015019 ; DE19939690C2; DE 60028958T2 ; DE 10 2004 026152 A1.

WO2011082378A2 relates to a process for the purification of succinic acid from a fermentation broth containing ammonium succinate. The process for the purification of succinic acid described in this invention involves the use of ion exchange resins for splitting the ammonium succinate in the fermentation broth. During the passage of the fermentation broth through a cationic ion exchange resin, the ammonium succinate is split into ammonium cation and the succinate anion. The proton on the resin surface is exchanged for the ammonium ions and the succinate anion is reduced to succinic acid with the protons released from the ion exchange resin. The bound ammonium is released from the resin with the addition of a strong acid such as sulfuric acid and thereby the ion exchange resin is regenerated for subsequent use. The ammonium sulfate by-product resulting from the regeneration step of this process can be used as a source of fertilizer. This process for the separation of succinic acid from the fermentation broth containing ammonium succinate can also be carried out with an anionic ion exchange resin wherein the succinate anion is retained on the surface of the ion exchange resin and subsequently released from the ion exchange resin during the regeneration step.

Furthermore a multitude of methods pertaining to the purification of lactic acid are known.

As an example, several patents teach that distillation be used for the purification of lactic acid from aqueous solutions. EP 0986532 B2 avails itself of this type of to process. DE 10 2007 045 701 B3 reveals a combined extraction with linear n-trioctylamine (TOA) and distillation. Other possibilities known from specialist literature are electrodialysis and/or esterification with an alcohol, whereby, in this case too, distillation and then hydrolysis are carried out on the ester formed. These processes are extremely expensive. In addition, a disadvantage of distillation is that part of the carbohydrate is always extracted as well which leads to a deterioration in the yield for the whole process and makes it more difficult to isolate the product.

Processes using calcium hydroxide and sulphuric acid are also known, wherein gypsum is produced in large quantities as a by-product. In this context it was also found that the lactic acid from a fermentation broth acidified with sulphuric acid, for example, which alongside free lactic acid still contains ammonium and sulphate ions, can be isolated using chromatographic methods. DE 69815369 T2 describes, for example, amongst others the separation of lactic acid from aqueous mixtures by means of adsorption into a solid adsorbent, preference being given here to the use of a solid adsorbent which adsorbs lactic acid versus lactate. According to the above-mentioned specification, weak anion exchangers in particular can be considered for isolating lactic acid. Furthermore, DE 10 2009 019 248 A1 describes chromatographic methods for the purification of organic acids, particularly for lactic acid, wherein a simulated moving bed chromatography is carried out.

The disadvantage with many processes is that additional substances are added to the process which may not be contained in the target product, and/or any traces of them in the target product can lead to restrictions in the quality and application of the product. Moreover, to some extent the practical execution of the process also involves considerable technical investment and energy consumption.

### SUMMARY OF THE INVENTION

Disclosed herein is a process available for separating and purifying carboxylic acids from fermentation broths, which may avoid the known disadvantages of other processes. The desired process should also supply an apparatus for carrying out the related process and a use for the current process.

### DETAILED DESCRIPTION

Disclosed herein, in one embodiment of the invention, is a process for the purification of carboxylic acid having a chain length from one to five carbon atoms comprising the steps of: (a) transforming the carboxylic acid to its monoester and/or its diester, and (b) processing the mono- and/or the diester of the carboxylic acid by subcritical or supercritical fluid chromatography using a subcritical or supercritical mobile phase.

Subcritical or supercritical fluid chromatography (SFC) is a separation process where mostly carbon dioxide below or above critical pressure and temperature is used as mobile phase. Mobile phase carries sample through the column, which is packed with stationary phase. Separation of several compounds is possible due to different adsorption potentials of compounds, meaning different interaction between compound molecules and surface of stationary phase. Interactions are presented as dipole, induced dipole or as H-bond. Adsorption potential is energy required for breaking interactions between compounds and surface of stationary phase. Therefore, solvent strength of mobile phase (solubility of compounds in mobile phase) has to be large enough in order to prevent long term adsorption. Solvent strength and other properties of carbon dioxide could be manipulated with pressure and temperature changing.

In case of separation of polar compounds pure non-polar carbon dioxide should be modified with polar modifiers such as ethanol and methanol, to enhance solvent strength.

Advantages of SFC include the use of compressible fluids, which are gaseous at atmospheric pressure; therefore products do not contain any solvent residues. Solubility of compounds in subcritical or supercritical fluids could be adjusted with altering density and vapor pressure, meaning changing pressure and temperature. The separation of amino acids using SFC technology is disclosed in Nogle L. M. et al.:“Preparative separation and identification of derivatized beta-methylphenylalanine enantiomers by chiral SFC, HPLC and NMR for development of new peptide ligand mimetics in drug discovery”, Journal of Pharmaceutical and Biomedical Analysis, New York, N.Y., US, vol. 40, no. 4,3 March 2006, pages 901-909. The purification of fatty acids using to supercritical fluid chromatography and subjecting them to SFC is disclosed in WO 2007/147554 A2. But in those documents the substances that have to be separated were soluble in supercritical fluid, contrary to this invention, where carboxylic acids having a chain length from one to five carbon atoms are practically supercritical fluid insoluble and therefore have to be transformed in supercritical fluid soluble substances before.

In order to perform process step (a), synthesis of esters are well known in the state of the art and described for example in U.S. Pat. No. 1,400,852. Also some recent publications describe the transformation of succinic acid to esters as purification process (Orjuela, A., Abraham J., Yanez, A. J., Peereboom, L., Lira, C. T., Miller, D. J., A novel process for recovery of fermentation-derived succinic acid, Separation and Purification Technology, (2011), 83,31-37. and Orjuela, A., Kolah, A., Hong, X., Lira, C. T., Miller, D. J. Diethyl succinate synthesis by reactive distillation, Separation and Purification Technology, (2012), 88, 151-162.).

Also U.S. Pat. No. 6,291,708B1 describes a process for producing an organic acid and optionally for simultaneously producing an ester of the organic acid is disclosed. The process comprises the steps of: (a) combining an aqueous diluent, an ammonium salt of an organic acid, and an alcohol, thereby forming a homogeneous liquid feed mixture; (b) rapidly heating the feed mixture at a pressure sufficient to suppress at least some vaporization of the alcohol and holding it at a temperature and for a time sufficient to decompose the ammonium salt of the organic acid into ammonia and free organic acid while rapidly removing the ammonia from the reaction-mass transfer equipment, and optionally to react at least some of the free organic acid with the alcohol to form an ester of the organic acid, thereby producing (i) a vapor product stream that comprises ammonia, water, and alcohol, and (ii) a liquid product stream that comprises free organic acid, optionally ester, and alcohol, where of the total quantity of alcohol in the vapor product stream and the liquid product stream, at least about 10% by weight is present in the liquid product stream; and (c) recovering the free organic acid and optionally the ester from the liquid product stream. The liquid feed mixture can comprise a concentrated crude or partially purified broth produced by a fermentation process.

In an embodiment of the invention process step b) may be performed in a to pressure range from 1 bar to 1000 bar, and may be preferably performed in a pressure range from 10 bar to 500 bar. The process may be performed in a temperature range from 0° C. to 200° C., and may be preferably performed in a temperature range from 5° C. to 80° C.

The stationary phase of the subcritical or supercritical fluid chromatography may be performed at a chiral or an achiral stationary phase. In an embodiment of the inventive process the achiral stationary phase is based on silica dioxide. To the stationary phase, functional groups are attached. The functional groups are selected from a group comprising hydroxyl groups, fluorophenyl functional groups, cyano groups, amino functional groups, amide functional groups, chains of hydrocarbons with 1, 6, 8 or 18 carbon atoms, phenyl functional groups, molecules of glycerol reacted with silanol groups and chemically bonded 2-ethylpyridine. The functional groups are especially selected from a group comprising 2- and 4-ethylpyridine, dipyridyl, dicyanoimidazole, morpholine, propylacetamide, benzamide, methanesulfonamide, benzenesulfonamide, 4-fluorobenenesulfonamide, 4-nitrobenenesulfonamide, diethylaminopropyl and 3-aminopropyl-N-dinitrotoluene.

A number of silica-based amide, urea, sulfonamide and pyridine containing stationary phases are commercially available.

In a further embodiment the chiral stationary phase is based on oligosaccharides selected from the group comprising cellulose or cyclodextrin. For these columns CHIRSLPAK IA (derivative of amylase), CHIRALPAK IB and CHIRALPAK IC (derivative of cellulose), CHIRALPAK AD® and CHIRALPAK AS® CHIRALCEL OD and OJ, Ion exchange columns and ligand exchange columns can be used.

Furthermore process step b) may be performed by selecting the subcritical or supercritical mobile phase from a group comprising carbon dioxide, nitrous oxide, propane, sulphur hexafluoride, ethane and mixtures thereof.

The carboxylic acid to be purified is selected from a group comprising lactic acid, succinic acid, acetic acid, fumaric acid, malic acid and maleic acid.

In case the carboxylic acid is desired the mono- and/or the diester of the carboxylic acid is hydrolyzed back to the carboxylic acid by methods known in the state of the art.

The invention also relates to a device for carrying out the mentioned process. The apparatus for the purification of carboxylic acid having a chain length from one to five carbon atoms typically comprises
a high pressure reactor suitable for operating at pressures from 1 to 500 bar at temperatures from 15 to 150° C. for transformation of carboxylic acid into its mono and/or its diesters in a water medium, and
a chromatographic apparatus where the mono and/or diester of carboxylic acid can be processed by subcritical or supercritical fluid chromatography using a subcritical or supercritical mobile phase.

Further on, the subcritical or supercritical chromatography comprises a detector selected from the group comprising a UV-VIS spectrophotometric detector, diode array UV detector, infrared spectrophotometric detector with high pressure cell, flame ionization detector and evaporative light scattering detectors.

In an embodiment of the current invention, the subcritical or supercritical chromatography also includes a mass spectrometer.

Using the current invention may generate carboxylic acids of a purity of approximately 97-99.9%.

In the following the current invention is described by way of example.

### EXAMPLE 1

A solid mixture of succinic acid and maleic acids was transferred to monomethyl and dimethyl esters of acids. Chromatographic separation was performed on pilot scale unit operated at 320 bar and temperature 40° C. and is equipped with FID detector (temperature 275° C.). The mobile phase was pure carbon dioxide and the flow rate was 150 kg/h. As a stationary phase the Lichrospherer 100, Chromsep, RP8 with dimension to 800 mm×100 mm, particle size 5 μm was used. The separation of monomethyl esters as well of dimethyl esters was very efficient and the products with purity of 99.1% could be obtained.

### EXAMPLE 2

A solid mixture of succinic acid and lactic acids was transferred to monomethyl and dimethyl esters of their acids. Chromatographic separation was performed on pilot scale unit operated at 300 bar and temperature 60° C. and is equipped with FID detector (temperature 275° C.). The mobile phase was pure carbon dioxide and the flow rate was 200 kg/h. As a stationary phase the Lichrospherer 100, Chromsep, RP8 with dimension 850 mm×100 mm, particle size 5 μm was used. The separation of monomethyl esters as well of dimethyl esters was very efficient and the products with purity of 98.9% could be obtained.

### EXAMPLE 3

A solid mixture of succinic acid and pyruvic acids was transferred to monomethyl and dimethyl esters of acids. Chromatographic separation was performed on pilot scale unit operated at 310 bar and temperature 55° C. and is equipped with FID detector (temperature 275° C.). The mobile phase was pure carbon dioxide and the flow rate was 150 kg/h. As a stationary phase the Lichrospherer 100, Chromsep, RP8 with dimension 800 mm×100 mm, particle size 5 μm was used. The separation of monomethyl esters as well of dimethyl esters was very efficient and the products with purity of 99.7% could be obtained.

### EXAMPLE 4

A solid mixture of succinic acid and maleic acid was transferred to monomethyl and dimethyl esters of acids. Chromatographic separation was performed on pilot scale unit operated at 150 bar and temperature 40° C. and is equipped with FID detector (temperature 275° C.). The mobile phase was pure propan and the flow rate was 80 kg/h.

As a stationary phase the Lichrospherer 100, Chromsep, RP8 with dimension 800 mm×100 mm, particle size 5 μm was used. The separation of monomethyl esters as well of dimethyl esters was very efficient and the products with purity of 98.7% could be obtained.

### EXAMPLE 5

A solid mixture of succinic acid and maleic acid was transferred to monomethyl and dimethyl esters of acids. Chromatographic separation was performed on pilot scale unit operated at 290 bar and temperature 80° C. and is equipped with ELSD detector. The mobile phase was pure carbon dioxide and the flow rate was 150 kg/h. As a stationary phase the CHIRALCEL OD with dimension 830 mm×100 mm, particle size 10 μm was used. The separation of monomethyl esters as well of dimethyl esters was very efficient and the products with purity of 99.3% could be obtained.

### EXAMPLE 6

A solid mixture of succinic acid and pyruvic acids was transferred to monomethyl and dimethyl esters of acids. Chromatographic separation was performed on pilot scale unit operated at 820 bar and temperature 110° C. and is equipped with MS detector. The mobile phase was mixture propane/carbon dioxide (mass ratio 1:1) and the flow rate was 200 kg/h. As a stationary phase the CHIRALCEL OD with dimension 830 mm×100 mm, particle size 10 μm was used. The separation of monomethyl esters as well of dimethyl esters was very efficient and the products with purity of 99.0% could be obtained.

### EXAMPLE 7

A mixture of lactic acid and acetic acids was transferred to monomethyl and dimethyl esters of acids. Chromatographic separation was performed on pilot scale unit operated at 280 bar and temperature 20° C. and is equipped with ELSD detector. The mobile phase was pure sulfurhexafluoride and the flow rate was 195 kg/h. As a stationary phase the CHIRALCEL OD with dimension 560 mm×100 mm, particle size 10 μm was used. The separation of monomethyl esters as well of dimethyl esters was very efficient and the products with purity of 99.2% could be obtained.

### EXAMPLE 8

A mixture of lactic acid and maleic acids was transferred to monomethyl and dimethyl esters of acids. Chromatographic separation was performed on pilot scale unit operated at 423 bar and temperature 60° C. and is equipped with ELSD detector. The mobile phase was pure sulfurhexafluoride and the flow rate was 295 kg/h. As a to stationary phase the silica modified with 2- and 4-Ethylpyridine with dimension 670 m×100 mm, particle size 5 μm was used. The separation of monomethyl esters as well of dimethyl esters was very efficient and the products with purity of 99.0% could be obtained.

### EXAMPLE 9

A mixture of lactic acid and succinic acids was transferred to monomethyl and dimethyl esters of acids. Chromatographic separation was performed on pilot scale unit operated at 370 bar and temperature 95° C. and is equipped with ELSD detector. The mobile phase was pure carbon dioxide and the flow rate was 195 kg/h. As a stationary phase the silica modified with 2- and 4-Ethylpyridine with dimension 600 mm×100 mm, particle size Sum was used. The separation of monomethyl esters as well of dimethyl esters was very efficient and the products with purity of 96.8% could be obtained.

### EXAMPLE 10

A mixture of lactic acid and succinic acids was transferred to monomethyl and dimethyl esters of acids. Chromatographic separation was performed on pilot scale unit operated at 277 bar and temperature 35° C. and is equipped with MS detector. The mobile phase was pure carbon dioxide and the flow rate was 195 kg/h. As a stationary phase the silica modified with fluorophenyl functional groups with dimension 700 mm×100 mm, particle size 5 μm was used. The separation of monomethyl esters as well of dimethyl esters was very efficient and the products with purity of 97.9% could be obtained.

1.-13. (canceled)

14. A process for the purification of carboxylic acid having a chain length from one to five carbon atoms comprising the steps of:
a) transforming the carboxylic acid to at least one of its monoester and its diester, and
b) processing the at least one of the mono- and the diester of the carboxylic acid by subcritical fluid chromatography using a subcritical mobile phase.

15. The process according to claim 14, wherein process step b) is performed in a pressure range from 1 bar to 1000 bar and a temperature range from 0° C. to 200° C.

16. The process according to claim 14, wherein process step b) is performed in a pressure range from 10 bar to 500 bar.

17. The process according to claim 14, wherein process step b) is performed in a temperature range from 5° C. to 80° C.

18. The process according to claim 14, wherein process step b) is performed at a chiral stationary phase based on oligosaccharides selected from the group consisting of:
cellulose and cyclodextrin.

19. The process according to claim 14, wherein process step b) is performed at an achiral stationary phase based on silica dioxide.

20. The process according to claim 19, wherein at least one functional group is attached to the stationary phase, and wherein the functional group is selected from a group consisting of: hydroxyl groups, fluorophenyl functional groups, cyano groups, amino functional groups, amide functional groups, chains of hydrocarbons with 1, 6, 8 or 18 carbon atoms, phenyl functional groups, molecules of glycerol reacted with silanol groups, and chemically bonded 2-ethylpyridine.

21. The process according to claim 20, wherein the at least one functional group is selected from the group consisting of 2- and 4-ethylpyridine, dipyridyl, dicyanoimidazole, morpholine, propylacetamide, benzamide, methanesulfonamide, benzenesulfonamide, 4-fluorobenenesulfonamide, 4-nitrobenenesulfonamide, diethylaminopropyl, and 3-aminopropyl-N-dinitrotoluene.

22. The process according to claim 14, wherein process step b) is performed by selecting the subcritical mobile phase from a group consisting of carbon dioxide, nitrous oxide, propane, sulphur hexafluoride, ethane, and mixtures thereof.

23. The process according to claim 14, wherein the carboxylic acid to be purified is selected from a group consisting of lactic acid, succinic acid, acetic acid, fumarid acid, malic acid, and maleic acid.

24. A process for the purification of carboxylic acid having a chain length from one to five carbon atoms comprising the steps of:
a) transforming the carboxylic acid to at least one of its monoester and its diester, and
b) processing the at least one of the mono- and the diester of the carboxylic acid by supercritical fluid chromatography using a supercritical mobile phase.

25. The process according to claim 24, wherein process step b) is performed at a chiral stationary phase based on oligosaccharides selected from the group consisting of:
cellulose and cyclodextrin.

26. The process according to claim 24, wherein process step b) is performed at an achiral stationary phase based on silica dioxide.

27. The process according to claim 26, wherein at least one functional group is attached to the stationary phase, and wherein the functional group is selected from a group consisting of: hydroxyl groups, fluorophenyl functional groups, cyano groups, amino functional groups, amide functional groups, chains of hydrocarbons with 1, 6, 8 or 18 carbon atoms, phenyl functional groups, molecules of glycerol reacted with silanol groups, and chemically bonded 2-ethylpyridine.

28. The process according to claim 27, wherein the at least one functional group is selected from the group consisting of 2- and 4-ethylpyridine, dipyridyl, dicyanoimidazole, morpholine, propylacetamide, benzamide, methanesulfonamide, benzenesulfonamide, 4-fluorobenenesulfonamide, 4-nitrobenenesulfonamide, diethylaminopropyl, and 3-aminopropyl-N-dinitrotoluene.

29. The process according to claim 24, wherein process step b) is performed by selecting the supercritical mobile phase from a group consisting of carbon dioxide, nitrous oxide, propane, sulphur hexafluoride, ethane, and mixtures thereof.

30. The process according to claim 24, wherein the carboxylic acid to be purified is selected from a group consisting of lactic acid, succinic acid, acetic acid, fumaric acid, malic acid, and maleic acid.

31. An apparatus for the purification of carboxylic acid having a chain length from one to five carbon atoms comprising:
a high pressure reactor suitable for operating at pressures from 1 to 500 bar at temperatures from 15 to 150° C. for transformation of carboxylic acid into at least one of its mono- and diesters in a water medium; and
a chromatographic apparatus wherein the at least one mono and diester of carboxylic acid is processed by at least one of:
(a) subcritical fluid chromatography using a subcritical mobile phase; and
(b) supercritical fluid chromatography using a supercritical mobile phase.

32. The apparatus of claim 31, wherein the chromatographic apparatus comprises a detector selected from the group consisting of: a UV-VIS spectrophotometric detector, diode array UV detector, infrared spectrophotometric detector with high pressure cell, flame ionization detector, and evaporative light scattering detectors.

33. The apparatus of claim 31, wherein the chromatographic apparatus comprises a mass spectrometer.
